SEQUENCE LISTING (6 sequences)

SEQ ID NO: 1 - DNA, 21 bp, Artificial Sequence
  Description of Artificial Sequence Synthetic primer
aagtgttgcccctgcaggaat

SEQ ID NO: 2 - DNA, 21 bp, Artificial Sequence
  Description of Artificial Sequence Synthetic primer
gagtggaactgctgggccata

SEQ ID NO: 3 - DNA, 22 bp, Artificial Sequence
  Description of Artificial Sequence Synthetic primer
aagcagctggctctacaaagca

SEQ ID NO: 4 - DNA, 19 bp, Artificial Sequence
  Description of Artificial Sequence Synthetic primer
agcatcctttggggcttca

SEQ ID NO: 5 - DNA, 52 bp, Artificial Sequence
  Description of Artificial Sequence Synthetic oligonucleotide
gactgactgactgactgactgactgactgactgacttgtggggctccctaga

SEQ ID NO: 6 - DNA, 63 bp, Artificial Sequence
  Description of Artificial Sequence Synthetic oligonucleotide
gactgactgactgactgactgactgactgactgactgactgactgactgcctgctgggagcct

(19) United States
(12) Patent Application Publication
(10) Pub. No.: US 2008/0275728 A1
(43) Pub. Date: Nov. 6, 2008

(54) Method for Personalized Diet Design for Females of Asian Origin

(75) Inventor: Jose M. Ordovas, Framingham, MA (US)

Correspondence Address: DAVID S. RESNICK, NIXON PEABODY LLP, 100 SUMMER STREET, BOSTON, MA, 02110-2131, US

(73) Assignee: TRUSTEES OF TUFTS COLLEGE, Medford, MA (US)

(21) Appl. No.: 12/089,003

(22) PCT Filed: Oct. 5, 2006
(86) PCT No.: PCT/US06/38982

§ 371 Date: Apr. 2, 2008

Related U.S. Application Data
(60) Provisional application No. 60/723,733, filed on Oct. 5, 2005.

(51) Int. Cl. G06Q 50/00; C12Q 1/68

(52) U.S. Cl. 705/2; 435/6

(57) ABSTRACT

The present invention provides methods for personalized diet design for females of Asian origin, wherein females who are homozygous for the PLIN 11482A are advised to avoid diets high in fat and low in carbohydrates, to avoid or manage metabolic syndrome and conditions related to metabolic syndrome, such as insulin resistance, diabetes, and cardiovascular disease.

### CROSS-REFERENCE TO RELATED APPLICATIONS

This application claims benefit under 35 U.S.C. 119(e) of U.S. application Ser. No. 60/723,733, filed Oct. 5, 2005, the content of which is herein incorporated by reference in its entirety.

### GOVERNMENT SUPPORT

This invention was made, at least in part, with the U.S. Government Support under Grant No. HL54776 awarded by NIH/NHLBI aware, and contracts 53-K06-5-10 and 58-1950-9-001 and 58-3148-2-083 from the US Department of Agriculture, the U.S. Government has certain rights thereto.

### BACKGROUND OF THE INVENTION

The association between fat or carbohydrate (CHO) content of the diet and insulin resistance and weight regulation is currently generating a wide-degree of public interest as well as important academic debate, both in Asian and in White populations (21-26). For several decades, medical societies and government agencies have been promoting high CHO diets as part of a healthy diet aimed to prevent chronic diseases. However, more recently, low CHO diets have been on the rise fostered by the notion that reduction in CHO consumption will result in a lower basal insulin concentration, hence promoting more triacylglycerol lipolysis into free fatty acids and leading to fat loss in obese subjects (27).

However, although there are some studies showing that low-CHO, high-fat diets improve insulin sensitivity and weight reduction in obese patients (28, 29), there is insufficient evidence to make general recommendations for or against the use of low-CHO diets (23-24).

Moreover, the vast majority of published studies have not taken into account the genetic characteristics of participants. Therefore, based on the enticing evidence supporting that genetic variation has an important influence in modulating the dietary effect on phenotyphical traits (30), it is reasonable to assume that the effects of macronutrient dietary composition on insulin resistance measures may be different depending on the individual genotype.

However, the search of relevant polymorphisms in candidate genes that interact with dietary intake is not an easy task as shown by the inconsistencies seen in the literature. In this regard, although the common variant Pro12Ala in the nuclear receptor peroxisome proliferator-activated receptor-gamma (PPARgamma) was initially reported to interact with the ratio of dietary polyunsaturated fatty acids (PUFA) to monounsaturated fatty acids (SFA) in determining fasting insulin and body-mass-index (BMI) (31), subsequent studies (32, 33) did not replicate the findings. Thus, the first report based the analysis of a White population (31) found that when the dietary PUFA to SFA ratio is low, the BMI and fasting insulin in alanine-carriers is greater than that in praline-homozygotes, but when the ratio is high, the opposite is seen. However, when we studied the Singaporean population (33), we did not find any significant interaction between dietary fat and the PPARgamma gene variation.

The prevalence of the metabolic syndrome has been steadily increasing worldwide during the past few decades, but the more dramatic quantitative increases are seen in some parts of Asia (1). Insulin resistance is one of the key elements involved in the metabolic syndrome and the subsequent increase in CVD risk (2, 3) that represents an increasing public health problem in South Asian populations (15). Socioeconomic development and abdominal obesity may be key underlying factors for the development of insulin resistance, the metabolic syndrome and subsequently, cardiovascular (CVD) disease risk. Two alternative models have been proposed to explain the common obesity observed in affluent populations (2). The first one, known as the absence of protection model is based on the assumption that the biological systems controlling energy homeostasis have evolved to protect against weight loss rather than weight gain resulting in obesity when there is an abundance of food. The second approach, known as the central resistance model, proposes the existence of biological defenses against pathological obesity; however, certain individuals have genetic and/or acquired resistance to adiposity regulating hormones that overrides this protection. Among several energy regulatory signals, leptin and insulin appear to be among the most serious candidates to serve as adiposity signals. Moreover, insulin signaling is essential for normal homeostasis of glucose, fat and protein metabolism. Therefore, dysregulation of this signaling, such as in insulin resistance, is associated with several disease-related phenotypes that have been clustered under the umbrella of the metabolic syndrome (3).

Accordingly, to improve management and treatment of metabolic disease, it would be useful to identify genetic markers that could provide guidance in diet design to reduce the risk factors in individuals.

### SUMMARY OF THE INVENTION

We have now discovered that genetic variation at PLIN locus is an important modulator of the effects of habitual dietary fat and CHO consumption on insulin resistance in a large sample of Asian women (Chinese, Malay and Indian) from the general population.

Accordingly, we have discovered that females of Asian origin, who are homozygotes for PLIN 11482A allele, or an allele in tight linkage disequilibrium with PLIN 11482A allele, have increased insulin resistance when consuming a high saturated fatty acids, low carbohydrate diet, whereas high percentage of carbohydrate intake showed protective effects. This gene-interaction effect was highly consistent among Chinese, Malay and Asian women showing that we have now found at least one relevant genetic marker to identify individuals with different susceptibility to macronutrient intake. Moreover, our findings add support to the notion that dietary recommendations to improve insulin resistance should move forward to more personalized guidelines that should include as part of their algorithms relevant genetic information. Accordingly, females of Asian origin, who are homozygotes for PLIN 11482A allele, should avoid consumption of low-CHO, high-SFA diets, such as Atkins diet, and the like, to avoid harmful health effects.

Accordingly, the invention provides a method for personalized diet design for females of Asian origin, wherein females who are homozygous for the PLIN 11482A are advised to avoid diets high in fat and low in carbohydrates, to avoid or manage metabolic syndrome and conditions related to metabolic syndrome, such as insulin resistance, diabetes, and cardiovascular disease.

### BRIEF DESCRIPTION OF THE FIGURES

FIG. 1 shows predicted values of the homeostasis model assessment-insulin resistance (HOMA-IR) in women from the Singapore population (Chinese, Malays and Asian Indians) according to the PLIN 11482G>A polymorphism (carriers of the 11482G allele, n=976; 11482 A homozygotes, n=177) plotted against the saturated fatty acids (SFA) to carbohydrate (CHO) ratio as a continuous variable. Predicted values (Ln) were calculated from the regression model containing SFA to CHO ratio intake, PLIN 11482G>A polymorphism, and their interaction term, after adjustment for ethnic group, age, BMI, cigarette smoking, alcohol consumption, physical activity, diabetes status, and energy intake. P-values indicate: The statistical significance of the interaction term in the adjusted regression model (P=0.002; the statistical significance of the adjusted regression coefficient in the regression line corresponding to PLIN 11482 A homozygotes (P<0.001); and the statistical significance of the adjusted regression coefficient in the regression line corresponding to carriers of the PLIN 11482G allele (P=0.719).

FIG. 2 shows predicted values of the homeostasis model assessment-insulin resistance (HOMA-IR) in Chinese women (A), Malay women (B) and Asian Indian (C) women according to the PLIN 11482G>A polymorphism (carriers of the 11482G allele: n=116 Chinese, n=196 Malays and n=182 Indians; 11482 A homozygotes: n=116 Chinese, n=43 Malays and n=18 Indians) plotted against the saturated fatty acids (SFA) to carbohydrate (CHO) ratio as a continuous variable. Predicted values (Ln) were calculated from the regression model containing SFA to CHO ratio intake, PLIN 11482G>A polymorphism, and their interaction term, after adjustment for age, BMI, cigarette smoking, alcohol consumption, physical activity, diabetes status, and energy intake. P-values indicate: The statistical significance of the interaction term in the adjusted regression model (P=0.012; P=0.063 and P=0.388, in A; B and C, respectively); the statistical significance of the adjusted regression coefficient in the regression line corresponding to PLIN 11482 A homozygotes (P=0.026; P=0.008 and P=0.242, in A; B and C, respectively); and the statistical significance of the adjusted regression coefficient in the regression line corresponding to carriers of the PLIN 11482G allele (P=0.745; P=0.465 and P=0.717, in A; B and C, respectively). When Malay and Indian women were analyzed together in the same multivariate adjusted model, the interaction term between SFA/CHO and the PLIN 11482G>A polymorphism was statistically significant (P=0.018). The P values for the regression coefficients were: P=0.005 in PLIN 11482A homozygotes, and P=0.442 in carriers of the PLIN 11482G allele.

### DETAILED DESCRIPTION OF THE INVENTION

The present invention is directed to creating dietary advise for individuals, particularly females of Asian origin.

In one embodiment, the individuals of Asian origin are selected from the group of individuals, particularly females for China, Singapore and/or Malaysia.

The invention is based on the finding that individuals, particularly females of Asian origin, who are homozygous for the PLIN 11482A allele are susceptible for metabolic syndrome and associated disease conditions, such as obesity, insensitivity to insulin, and consequent diabetes, when consuming diets low in carbohydrates and high in saturated fatty acids.

Alterations of normal adipocyte metabolism contribute to the development of insulin resistance. This may be mediated by the products of lipolysis or by adipokines. The relation between adipocyte metabolism and insulin resistance suggests that some of the genetic components involved in insulin resistance may be related to genes primarily expressed in adipocytes (4-5). In this regard, perilipin is the predominant protein associated with adipocyte lipid droplets. The key roles of perilipin in regulating lipid storage in adipocytes and the accumulation of body fat have been demonstrated in both in vitro and in vivo studies (6-9). Genetic variation at the perilipin gene (PLIN) has been associated with modulation of the perilipin content and lipolytic rate in humans (10). Consistent with those functional observations, we have found significant associations between genetic variants at this locus, body-weight and obesity risk in several ethnic groups (11-13); however, their relation to insulin resistance was not analyzed. The connection between perilipin, body fat and insulin resistance has been shown in knockout mouse models, which displayed reduced body fat and paradoxically an increased risk of glucose intolerance and peripheral insulin resistance (9). In addition, dietary factors play an important role in the relationship between perilipin and body-weight. Thus, in this same mouse model, ablation of PLIN expression was associated with differential sensitivity to obesity induced by a high-fat diet as compared with wild type mice (9). Along these lines, we found that a PLIN polymorphism modulate the weight loss in response to a low-energy diet (14).

Metabolic syndrome and conditions related thereto, as referred to herein and throughout the specification refers to condition, wherein at least one of the following symptoms is present: insulin resistance, type II (insulin independent) diabetes, triglyceride (TG) levels were equal or greater than the 90th percentile for age and sex, HDL equal or lower than 30 mg/dL for males and 34 mg/dL for females, and BMI ≦30 kg/m2; unaffected if their TG levels equal or lower the 50th percentile for their age and sex and high density lipoprotein (HDL) ≧37 mg/dL for men and ≧42 mg/dL for women and body-mass index (BMI) ≦30 kg/m2 and normoglycemic.

The PLIN 11482 polymorphism is analyzed from nucleic acids isolated from any biological sample taken from an individual. Biological sample used as a source material for isolating the nucleic acids in the instant invention include, but are not limited to solid materials (e.g., tissue, cell pellets, biopsies, hair follicle samples, buccal smear or swab) and biological fluids (e.g. blood, saliva, amniotic fluid, mouth wash, urine). Any biological sample from a human individual comprising even one cell comprising nucleic acid, can be used in the methods of the present invention.

Nucleic acid molecules of the instant invention include DNA and RNA, preferably genomic DNA, and can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001.

the PLIN 11482A/G polymorphisms according to the present invention can be detected from the isolated nucleic acids using techniques including direct analysis of isolated nucleic acids such as Southern Blot Hybridization (DNA) or direct nucleic acid sequencing (Molecular Cloning: A Laboratory Manual, 3rd Ed., Sambrook and Russel, Cold Spring Harbor Laboratory Press, 2001).

An alternative method useful according to the present invention for direct analysis of the PLIN 11482A/G polymorphisms is the INVADER® assay (Third Wave Technologies, Inc (Madison, Wis.). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535).

Preferably, a nucleic acid amplification, such as PCR based techniques are used. After nucleic acid amplification, the polymorphic nucleic acids can be identified using, for example direct sequencing with radioactively or fluorescently labeled primers; single-stand conformation polymorphism analysis (SSCP), denaturating gradient gel electrophoresis (DGGE); and chemical cleavage analysis, all of which are explained in detail, for example, in the Molecular Cloning: A Laboratory Manual, 3rd Ed., Sambrook and Russel, Cold Spring Harbor Laboratory Press, 2001.

The PLIN 11482A/G polymorphisms are preferably analyzed using methods amenable for automation such as the different methods for primer extension analysis. Primer extension analysis can be preformed using any method-known to one skilled in the art including PYROSEQUENCING™ (Uppsala, Sweden); Mass Spectrometry including MALDI-TOF, or Matrix Assisted Laser Desorption Ionization—Time of Flight; genomic nucleic acid arrays (Shalon et al., Genome Research 6(7):639-45, 1996; Bernard et al., Nucleic Acids Research 24(8):1435-42, 1996); solid-phase mini-sequencing technique (U.S. Pat. No. 6,013,431, Suomalainen et al. Mol. Biotechnol. June; 15(2):123-31, 2000); ion-pair high-performance liquid chromatography (Doris et al. J. Chromatogr. A May 8; 806(1):47-60, 1998); and 5′ nuclease assay or real-time RT-PCR (Holland et al. Proc Natl Acad Sci USA 88: 7276-7280, 1991), or primer extension methods described in the U.S. Pat. No. 6,355,433. Nucleic acids sequencing, for example using any automated sequencing system and either labeled primers or labeled terminator dideoxynucleotides can also be used to detect the polymorphisms. Systems for automated sequence analysis include, for example, Hitachi FMBIO® and Hitachi FMBIO® II Fluorescent Scanners (Hitachi Genetic Systems, Alameda, Calif.); Spectrumedix® SCE 9610 Fully Automated 96-Capillary Electrophoresis Genetic Analysis System (SpectruMedix LLC, State College, Pa.); ABI PRISM® 377 DNA Sequencer; ABI® 373 DNA Sequencer; ABI PRISM® 310 Genetic Analyzer; ABI PRISM® 3100 Genetic Analyzer; ABI PRISM® 3700 DNA Analyzer (Applied Biosystems, Headquarters, Foster City, Calif.); Molecular Dynamics FluorImager™ 575 and SI Fluorescent Scanners and Molecular Dynamics FluorImager™ 595 Fluorescent Scanners (Amersham Biosciences UK Limited, Little Chalfont, Buckinghamshire, England); GenomyxSC™ DNA Sequencing System (Genomyx Corporation (Foster City, Calif.); Pharmacia ALF™ DNA Sequencer and Pharmacia ALFexpress™ (Amersham Biosciences UK Limited, Little Chalfont, Buckinghamshire, England).

Nucleic acid amplification, nucleic acid sequencing and primer extension reactions for one nucleic acid sample can be performed in the same or separate reactions using the primers designed to amplify and detect the polymorphic the PLIN 11482A/G nucleotides.

In one embodiment, the invention provides a kit comprising one or more primer pairs capable of amplifying the PLIN 11482 nucleic acid region; buffer and nucleotide mix for the PCR reaction; appropriate enzymes for PCR reaction in same or separate containers as well as an instruction manual defining the PCR conditions, for example, as described in the Example below.

The methods of the present invention include methods, wherein the actual polymorphism detection is performed by a third party anywhere in the world. Accordingly, the methods include taking a sample, and sending it to a third party to be genotyped, wherein the third party sends the genotype results to the provider institute or person, and wherein the individual in need of personalized diet received the advise either face to face, via a letter, telephone, e-mail, internet, extranet or via any other media or method. Also, the biological sample can be taken by the individual themselves, and be sent/supplied directly to the third party who is responsible to genotyping, or to the dietary advise service provider, who intends to deliver the results, and who will further provide the sample to the third party genotyping provider. Once the sample is genotyped, the results may also be sent automatically from the genotype provider or the third party, based on the genotype, i.e., if the individual is a female and of Asian origin, and is a homozygote for PLIN11482A allele, a system, such as a computer, can send proper diet guidelines including higher CHO and lower SFA intake instructions, instructions to avoid Atkins-like diets, and the like to the individual, whose sample was analyzed.

Primers may also be provided in the kit in either dry form in a tube or a vial, or alternatively dissolved into an appropriate aqueous buffer.

The kit may also comprise primers for the primer extension method for detection of the PLIN 11482G and/or A alleles.

In one embodiment, the components of the kit are part of a kit providing for multiple metabolic syndrome disease risk associated genes and polymorphisms and or mutations known to one skilled in the art, in addition to detecting PLIN 11482G and/or A polymorphisms. Such other mutations and/or polymorphisms include, but are not limited to mutations and polymorphisms associated with weight regulation.

In one preferred embodiment, the kit comprises a plurality of oligonucleotide probes on a solid surface for detecting the polymorphisms in PLIN 11482 locus. Preferably, the solid surface is a chip or a bead.

Diet with increased carbohydrates, which is recommended for the PLIN 11482A homozygotes, preferably comprises complex carbohydrates, and alternatively foods with low glycemic index.

The perilipin or PLIN11482 locus as used herein refers to PLIN4 at nucleotide 11482 of sequence with GenBank accession no. gi21431190.

### EXAMPLES

The tightness of the relationship between adipose metabolism and insulin resistance (IR) suggest one strategy to find candidate genes is to examine genes functionally relevant to adipocyte biology, such as the perilipin (PLIN) gene.

We decided to investigate the association between PLIN variation, and IR measures in a large Asian population, as well as to examine their modulation by dietary intake.

Cross-sectional study in 1909 males and 2198 females (aged 18-69 years of age) from the Singapore general population, including Chinese, Malays and Indians. Genetic (PLIN 11482G>A and 14995A>T polymorphisms), lifestyle, clinical and biochemical data were obtained. Homeostasis model assessment (HOMA-IR) was used to evaluate IR. Diet was measured by a validated food frequency questionnaire in a systematically selected (1 in 2) sample.

We did not find significant between-genotype difference in the IR among all the three ethnics (Chinese, Malay, and Indian) examined. However, in women, significant interactions were found between PLIN 11482G>A/14995A>T (as a recessive model) and saturated fat (SFA, P=0.003/0.004), and carbohydrate (CHO, P=0.004/0.012) in determining HOMA-IR, in opposite directions. Thus, females who carried homozygous 11482A, showed higher HOMA-IR with increasing intakes of SFA (P for trend among tertiles: 0.006). However, HOMA-IR decreased (P=0.046) as CHO intake increased. These effects were stronger when SFA and CHO were combined as a SFA to CHO ratio. No significant heterogeneity by ethnic group was observed.

Our objectives in this study were: 1) To estimate the association between genetic variation at the PLIN locus and insulin related measures and pathology in a large multiethnic Asian cohort, including Chinese, Malay and Indians, with different susceptibility to diabetes; and 2) To examine if dietary intake modulates the associations between the PLIN locus and measures of insulin resistance in this population.

### Subjects and Study Design

Participants were recruited in the framework of the 1998 Singapore National Health Survey (NHS 98). The detailed methodology of this survey of a nationally representative household sample has been described elsewhere (15). Briefly, the survey protocol was based on the WHO-recommended model for field surveys of diabetes and other noncommunicable diseases, and the WHO MONICA protocol for population surveys. Initially, 11,200 individuals from addresses representing the house-type (a proxy for socioeconomic status) distribution of the entire Singapore housing population were selected from the National Database on Dwellings. A process of disproportionate stratified and systematic sampling was used to select individuals between 18 and 69 years from this data set, with oversampling of the minority groups (Malays and Indians) to ensure that prevalence estimates for the minority groups were reliable. The ethnic composition of the sample ended up being 64% Chinese, 21% Malays and 15% Asian Indians and included 4723 men and women. The study was approved by the Ministry of Health in Singapore and informed consent was obtained from all participants. Among these participants, we included in our analyses 4107 subjects (1909 males and 2198 females) who had DNA available and PLIN genotypes successfully determined. Demographic, clinical, biochemical, genetic and lifestyle data were obtained in all these participants. Data on lifestyle factors were collected using an interviewer-administered questionnaire. Information on physical activity level was collected using an adaptation from the American College of Sports Medicine's classification. Alcohol consumption was assessed using a questionnaire based on the Behavior Risk Factor Surveillance Questionnaire from the Centers for Disease Control and Prevention. Daily smoker were defined as those who smoked more than one cigarette per day (15,16).

### Dietary Intake

To assess the habitual dietary intake, a previously validated food-frequency questionnaire (17) was administered to a random sub-sample of the participants. This questionnaire was previously validated in the Singaporean population against multiple 24-h recalls as well as urinary N excretion. At the beginning, subjects were systematically selected (1 in 2) to participate in the dietary survey. A food list of 159 individual food items grouped into 23 main food types and 25 sub-food types were included in this questionnaire. Each food group was carefully considered for ensuring the representatives of listed foods in the three ethnic groups. Nutrient contents were estimated using the food composition database of the Singapore Ministry of Health. Energy intake, carbohydrates (CHO), proteins, total fat, and specific fatty acids were computed for each individual. From these subjects, only those who had complete genetic, lifestyle, clinical al biochemical variables were included in the interaction analysis. In addition, to avoid the potential bias caused by the change in habitual dietary patterns in diabetic subjects who know their disease, dietary intake was only analyzed in the subjects who initially did not have diagnosis of diabetes. Thus, the gene-diet interaction analysis was carried out in 2154 participants (1001 men and 1153 women).

### Anthropometrical and Biochemical Measurements

Height was recorded in all subjects without shoes. Body weight was measured in subjects in light clothing using electronic weighing scales (SECA model 220). Body mass index (BMI) was computed using weight divided by the square of the height. Subjects were instructed to fast overnight for at least 10 h before a blood sample was collected (18). Total cholesterol, high-density lipoprotein cholesterol (HDL-C), triacylglycerol and low-density lipoprotein cholesterol (LDL-C) were determined as previously described (18). Fasting glucose and fasting insulin were determined in all participants. After the collection of the fasting sample, a 75 g oral glucose tolerance test (OGTT) was taken for all subjects except diabetics on medication (68 men and 65 women). A 75 g glucose dissolved in 250 mL of water was ingested. After 2 h, a second blood sample was obtained and glucose and insulin were measured. Glucose measurements were done using kits from Boehringer Mannheim (Boehringer Manheim Systems, Mannheim, Germany) and read on a BM/Hitachi 747 analyzer (Roche Diagnostics, Indianapolis, Ind.). Insulin was measured by microparticle enzyme immunoassay methods using an Abbot AxSYM (Chicago) insulin assay (intra-assay CV 4.1% and interassay CV 2.9%) in both fasting and after-challenge blood samples. Insulin resistance (IR) was calculated using the homeostasis model assessment (HOMA) method first proposed by Matthews et al. (HOMA-IR=fasting insulin (μU/mL)/[22.5xe-ln(glucose [mmol/L])] (19). Initially, previously diagnosed diabetes status was assessed by questionnaire. The most part of these subjects were taking medication of diabetes. After the biochemical determinations, in addition to these criteria, subjects with the fasting glucose level greater than or equal to 7.0 mmol/liter or the 2-hour post challenge glucose level greater than or equal to 11.1 mmol/liter were also classified as diabetic. Diagnostic criteria were in line with those recommended by the World Health Organization (20). Overweight/obesity status were defined according to the World Health Organization recommendations for BMI: overweight (25< or = to BMI <30 kg/m2), and obese (BMI > or =30 kg/m2).

### DNA Isolation and Genotyping

DNA was isolated from blood samples and PLIN polymorphisms were determined by multiplex polymerase chain reaction (PCR). Two common PLIN polymorphisms (PLIN 11482G>A and PLIN 14995A>T) were genotyped. These polymorphisms were selected because in our previous work in which we studied the intragenic linkage disequilibrium (LD) and structure of the human PLIN gene (13) we found that the PLIN 14995A>T SNP was the most informative single genetic marker and that this polymorphism was in strong linkage with the 11482G>A SNP. Briefly, the DNA fragments encompassing polymorphisms were amplified by multiplex polymerase chain reaction (PCR). The primers used were: Forward, AAGTGTTGCCCCTGCAGGAAT(SEQ ID NO: 1 and Reverse, GAGTGGAACTGCTGGGCCATA (SEQ ID NO: 2) for the PLIN 11482G>A, and: Forward, AAGCAGCTGGCTCTACAAAGCA (SEQ ID NO: 3) and Reverse: AGCATCCTTTGGGGCTTCA (SEQ ID NO: 4) for the PLIN 14995A>T. PCR amplification was carried out in a 10 μl reaction volume containing 0.2 mmol/l of each dNTP, 0.2 μmol/l of each primer, 3.0 mmol/1 magnesium chloride, and 0.8 U of Qiagen Hotstar Taq polymerase. PCR cycling conditions were as previously described (13). The PCR products were incubated for 60 min at 37° C. with 2.5 U each of Exonuclease I (New England Biolabs., Inc. Beverly, Mass.) and Calf Intestinal Phosphatase (New England Biolabs., Inc. Beverly, Mass.) to remove un-incorporated dNTPs and primers. This was followed by incubation for 15 min at 75° C. to inactivate the enzymes. Single Nucleotide Extension was then carried using the ABI Prism SnaPshot multiplex system (Applied Biosystems, Foster City, Calif.). Probes used for Single Nucleotide Extension were: GACTGACTGACTGACTGACTGACTGACTG-ACTGACTTGTGGGGCTCCCTAGA (SEQ ID NO: 5) and GACTGACTGACTGACTGACTGACTGACTGACTGACTGACTGACTGACTGCCTG CTGGGAGCCT (SEQ ID NO: 6) for the 11482G>A and the 14995A>T SNPs, respectively. The extension reaction was carried out using PCR thermocycler in a 5 μl reaction mixture as previously described (13). Genotyping was carried with the final products on an ABI Prism 3100 genetic analyzer (Applied Biosystems, Foster City, Calif.) using Genotyper version 3.7 (Applied Biosystems, Foster City, Calif.).

### Statistical Analyses

The estimation for allele frequency, the test of the deviation of genotypes for each polymorphism from Hardy-Weinberg equilibrium, and the estimation for pair-wise LD between SNPs were carried out using genetic data analysis programs Arlequin (Schneider, S., Roessli, D., and Excoffier, L. (2000) Arlequin: A software for population genetics data analysis. Ver 2.000. Genetics and Biometry Lab, Dept. of Anthropology, University of Geneva). The statistical significance of pairwise LD was tested using a likelihood-ratio test. Chi-squared test were used to test the association between categorical variables. General linear regression model was used to analyze the association between the genotypes at each polymorphic site and measures of insulin sensitivity. Analyses were adjusted for age, BMI, ethnicity, cigarette smoking, alcohol consumption and exercise. When dietary variables were examined as independent variables, additional adjustment for total energy intake and other macronutrients was considered. Dietary fat and CHO intake were first treated as categorical according to the population tertiles. In addition, the SFA to CHO ratio was computed and treated as a continuous variables. A general inheritance model (subjects were grouped according to the genotypes of each SNP) was initially fitted, and, appropriate inheritance models (dominant, recessive, or additive) were finally used based on observed allelic effects. ANCOVA was employed to compare phenotypic outcomes between genotypic groups with adjustments for the covariates described above. The interactions between genetic effect and dietary factors were analyzed by introducing the corresponding product terms into the models. When dietary intake was considered to be a continuous variable, its interaction with the PLIN polymorphisms was depicted by computing the predicted values for each individual from the adjusted regression model and plotting these values against dietary intake depending on the PLIN genotype. Logarithm transformation was carried for fasting glucose, fasting insulin, post-challenge insulin, OGTT, and HOMA-IR before statistical testing to improve the normality. Standard regression diagnostic procedures were used to ensure the appropriateness of the regression models. SAS (Windows version 8.0) was used to analyze the data. All reported probability tests were two-sided.

### Results

The general characteristics of the 4107 participants by gender and ethnic group (2763 Chinese, 746 Malays and 598 Asian Indians) are presented in Table 1. Initially, 147 subjects (77 men and 70 women) reported that they were previously diagnosed as diabetic. From those, 68 men (34 Chinese, 13 Malays and 21 Asian Indians) and 65 women (24 Chinese, 22 Malays and 22 Asian Indians) were on diabetes medication. Biochemical data (fasting glucose and OGTT) obtained during the study revealed that 245 subjects (105 men and 140 women) could be adjudicated as newly diagnosed diabetic. Thus, 392 participants (182 men and 210 women) were finally classified as diabetic subjects. Prevalence of diabetes was statistically higher in Indians, followed by Malays and Chinese (P<0.001). The distributions of both PLIN 11482G>A and PLIN 14995A>T genotypes did not deviate from Hardy-Weinberg equilibrium in the entire cohort or in each ethnic group. Allele frequencies for the less common alleles did not differ by gender in any ethnic group (P>0.05). For both polymorphisms, frequencies of the less common alleles were statistically (P<0.01) lower in Asian Indians (0.33 and 0.36 for PLIN 11482G>A and PLIN 14995A>T, respectively) than in the other groups (0.42 and 0.44 for PLIN 11482G>A and PLIN 14995A>T, respectively in Chinese; and 0.44 and 0.44 for PLIN 11482G>A and PLIN 14995A>T, respectively in Malays). There was strong (D′ ˜0.80, P<0.0001) pair-wise LD between PLIN 11482G>A and PLIN 14995A>T across all three ethnic groups.

### Gene-Phenotype Associations

We first examined the association between the PLIN 11482G>A and 14995A>T polymorphisms and diabetes (both the previously reported and the newly diagnosed). We did not find any significant association between these polymorphisms and prevalence of diabetes in any ethnic group or gender. Then, we studied the potential association between these polymorphisms and variables related with insulin sensitivity and carbohydrate metabolism (fasting glucose, OGTT, fasting insulin, post-challenge insulin, and HOMA-IR) by ethnic group and gender. As observed before for the diabetes phenotype, we did not obtain any significant association between these polymorphisms and biochemical variables across ethnics groups. Table 2 shows adjusted means (for age, BMI, diabetes, cigarette smoking, alcohol consumption, and exercise) of fasting glucose, OGTT, fasting insulin, post-challenge insulin, and HOMA-IR) by ethnic group and gender depending on the PLIN 11482G>A polymorphism.

### Gene-Diet Interaction in Determining Insulin-Resistance

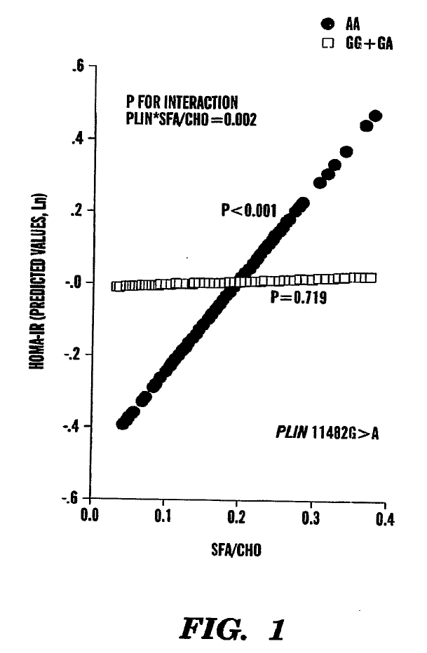

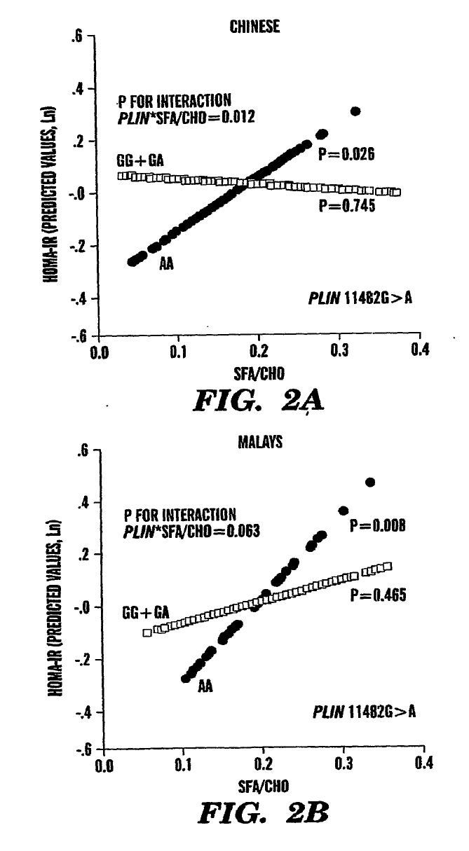

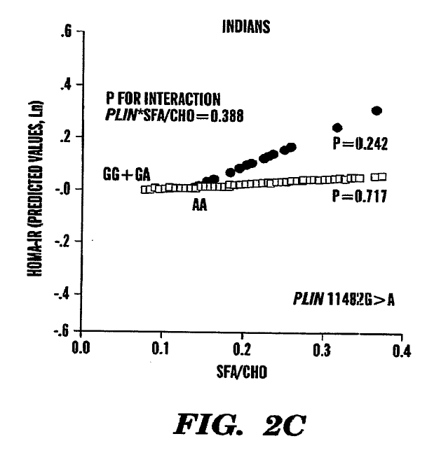

We then analyzed whether macronutrient dietary intake modulates the association between PLIN 11482G>A and PLIN 14995A>T polymorphisms and insulin resistance-related measures. Dietary intake was measured in a random sample (1 in 2) of the participants. In addition, subjects who knew they were diabetic were excluded to remove the influence of previously diagnosed disease on dietary patterns. Table 3 shows dietary intake in these participants (n=2154) by ethnic group and gender. In analyzing the interaction between habitual dietary intake and PLIN polymorphisms, total fat, saturated fatty acids (SFA), monounsatured fatty acids (MUFA) and polyunsaturated fatty acids (PUFA), CHO and proteins were considered in the regression models. We did not find significant gene by ethnic group interactions and the analyses were carried out including all the three ethnic groups. Conversely, we found significant interaction with gender and we analyzed men and women separately. In addition, we observed a recessive effect of the variant allele; therefore, carriers of homozygous wild type allele were pooled together with those who were heterozygous (PLIN 11482G>A: GG+GA, and 14995A>T: AA+AT) and compared with subjects homozygous for the less common allele (AA and TT, respectively). Gender-specific tertiles of macronutrient intakes were calculated to test the interaction effects. In men, we did not find any significant interaction between any one of the PLIN polymorphisms and dietary macronutrients in determining insulin-resistance related variables. However, in women (n=1153) we found statistically significant interactions between the PLIN 11482G>A polymorphism and the tertiles of total fat intake, SFA, and CHO modulating fasting insulin concentrations (P=0.010, P=0.004, and P=0.007 respectively) and HOMA-IR (P=0.007, P=0.003, and P=0.004 respectively). Statistically significant interactions showing the same effects were observed for the PLIN 14995A>T polymorphism modulating fasting insulin level (P=0.014, P=0.012, and P=0.008) and HOMA-IR (P=0.012, P=0.005, and P=0.006) for total fat, SFA, and CHO respectively. Moreover, SFA showed significant interactions with PLIN 11482G>A (P=0.004) and PLIN 4995A>T (P=0.005) polymorphisms modulating fasting glucose. Due to the fact that both genetic variants were in strong LD and yielded the same results and to simplify data presentation, we only show results corresponding to the PLIN 11482G>A polymorphism (Table 4), The outcome of these gene-diet interactions can be summarized as follows: in women homozygous for the less common allele (AA for PLIN 11482G>A polymorphism; n=177), HOMA-IR increased as total fat, and specifically SFA, intake increased (P for lineal trend <0.015). Conversely, percent CHO intake was inversely correlated with HOMA-IR among women homozygous for the minor allele (P for lineal trend <0.05). Further adjustment of the SFA interaction by the CHO intake did not modify the statistically significance of the findings. Similarly, when the interaction between the PLIN 11482G>A polymorphism and CHO intake in determining insulin-resistance measures was adjusted for SFA, the interaction term remained statistically significant revealing independent and additive effects. Then, we carried out a combined analysis considering simultaneously intakes of SFA and CHO. The SFA to CHO ratio was calculated for each individual and the interaction term between this variable (as continuous) and the 11482G>A polymorphism was tested in a multivariate regression model after adjustment for ethnic group, age, BMI, diabetes, cigarette smoking, alcohol consumption, exercise and total energy intake. A statistically significant interaction was found in women (P=0.002) in determining HOMA-IR. FIG. 1 shows predicted values of HOMA-IR depending on the SFA/CHO intake and the PLIN 11482G>A polymorphism in Asian women. Clearly, as the SFA/CHO ratio increased, HOMA-IR in women homozygous for the 11482A allele increased (P<0.001 for the adjusted regression coefficient in the corresponding regression line). However, no significant increase in HOMA-IR was observed in women carrying the wild-type allele. Interestingly, no statistically significant heterogeneity of the increasing effect of SFA/CHO ratio on HOMA-IR in women homozygous for the 11482A allele among the three ethnic groups was detected, revealing a highly consistent effect across different degrees of insulin-resistance in the population. FIG. 2 shows predicted values of HOMA-IR in Chinese (A), Malay (B) and Indian (C) women depending on the SFA/CHO intake and the PLIN 11482G>A polymorphism. In all the three ethnic groups, HOMA-IR increased in women homozygous for the 11482A as the ratio SFA/CHO increased. This increase was statistically significant (P<0.05 for the corresponding regression coefficients) in Chinese and Malays, without reaching the statistical significance in Indian women because the lower prevalence of the 11482A allele in this ethnic group. To increase the statistical power and after having observed similar effects in both ethnic groups, Indian and Malay women were combined. When analyzed together, the interaction term between SFA/CHO and the PLIN 11482G>A was statistically significant (P=0.018) in determining HOMA-IR after controlling for covariates. Accordingly, the adjusted regression coefficient for the regression line corresponding to Malay and Indian PLIN 11482A homozygotes was statistically significant (P=0.005). Finally, further adjustment of the above regression models for protein intake did not modify the statistical significance of the findings.

In the present study, we have investigated the PLIN gene because of the relevant physiological role that the adipose tissue plays in insulin sensitivity and the metabolic syndrome (4, 5, 34, 35). PLIN is the predominant protein associated with lipid storage droplets in human adipocytes and one of the critical regulators implicated in the mobilization of lipid storage, in dynamic interplay with hormone-sensitive lipase (HSL) (6-10). In examining PLIN genetic variation, we have found a statistically significant, and strongly consistent, gene-diet interaction between the PLIN 11482G>A and the PLIN 14995A>T polymorphisms and dietary fat and CHO intake in determining insulin-resistance measures in women. As these polymorphisms were in strong linkage disequilibrium in all the three ethnic groups, and 80% of women were homozygyzotes for both the PLIN 11482A and the PLIN 14995T alleles at the same time, it was difficult to determine the separate effects of these polymorphisms. Then, we carried out a haplotype analysis considering both polymorphisms. Despite sample size limitations, this analysis suggested that PLIN 11482G>A polymorphism seems to be the most informative marker of the observed effects in this population.

It is interesting to highlight that when it comes to the reported dietary fat-gene interaction, the significance was observed only for SFA but no for MUFA or PUFA. This supports a large body of research in animal models and humans supporting the notion that SFA clearly worsen insulin-resistance, while PUFA and MUFA may improve it (36-37). Thus, in our study, women homozygous for the PLIN 11482A allele had higher insulin resistance measures when consuming increasing amount of SFA and decreasing amount of CHO. Although the reference measurement of insulin sensitivity is the hyperinsulinemic clamp (38), this method is impractical for use in large population studies, and alternative methods for measuring insulin resistance, including fasting plasma insulin and HOMA-IR, have therefore been validated (39-40). In the present study, we have measured both fasting and post-challenge glucose and insulin, as well as calculated the HOMA-IR score. In our hands, fasting insulin and the HOMA-IR were the most informative measures for the observed gene-diet interactions. According to our results, Asian women in the highest SFA tertile (11.8% to 19% of energy) had higher HOMA-IR (48% increase) than women in the lowest tertile (3.1% to 9.4% % of energy) only if they were homozygotes for the PLIN 11482G>A polymorphism. Conversely, PLIN 11482A homozygous women in the lowest CHO tertile had a 24% higher HOMA-IR score than women in the highest CHO tertile. These effects were independent and when combined intakes of SFA and CHO were considered, the differences were magnified. Such associations were not observed among women carrying the 11482G allele either for SFA or CHO intake. Our results clearly shown the effects of SFA and CHO content in the diet, on insulin resistance measures in Asian women largely depends on the genotype and may contribute to explain the inconsistent results found in different epidemiological studies. Thus, McKeown et al (41) in a study carried out in the Framingham Offspring Cohort including 2,834 subjects, found no association between total CHO intake and insulin resistance (HOMA-IR). Conversely, Lau et al (42) reported that total CHO were inversely related to HOMA-IR in a large sample of Danish men and women. Several epidemiological studies have found that total CHO intake is unrelated to fasting insulin (43, 44) and the risk of developing type 2 diabetes (45-47). Some has suggested that CHO intake is not a sensitive enough measure and the glycemic index (GI) has been proposed as a better measure to classify CHO-containing foods in terms of their glycemic effect (48). In the present study, we have not determined the GI precluding us from examining its interaction with PLIN variation. However, although a high dietary GI has been positively associated with HOMA-IR in some studies, uncertainties regarding its estimation reduce the GI validity in observational studies (49). Despite the widespread belief that SFA intake worsens cardiovascular disease risk related variables (50), inconsistency also exists when the association between SFA and insulin resistance is examined in epidemiological studies (25, 36, 44, 51) In the present study, SFA or the SFA to CHO ratio was not associated with insulin resistance measures in the population as a whole. On average, higher SFA and low CHO were strongly associated with higher HOMA-IR only in the subgroup of women (about 17%) who were homozygotes for the PLIN 11482G>A polymorphism. Interestingly, this gene-diet interaction was consistently found among Chinese, Malay and Asian women, despite their different susceptibilities to insulin resistance, increasing the level of causality of this statistical association. To the remarkable consistency of findings in women, we can add the consistent lack of statistically significant interactions observed in men regardless of ethnicity. Based on the current knowledge, this finding is not surprising. Previous studies carried out analyzing PLIN genetic variation have revealed gender-specific associations in women (11-13). Moreover, Mittendorfer has examined the evidence regarding sex differences in insulin resistance (52), and concluded that women are intrinsically more insulin resistant than males, possibly because of specific sex-linked gene expression and the resulting differences in metabolic control elements (e.g. signaling pathway and substrate shuttling elements, receptors). Furthermore, we have observed that in massively obese men, the effects of PLIN polymorphisms were similar than in women (14). The biological mechanisms underlying this gene-diet interaction remains to be determined; however, some experimental evidence supports our results. Thus, it has been reported that PLIN knockout mice, that were resistant to obesity induced by a high-fat diet, developed more insulin resistance than control mice (9). In addition, when studying the metabolic adaptation of PLIN-null mice (53), increased beta-oxidation and increased insulin resistance with age was observed by another independent group. Although the PLIN 11482G>A polymorphism is located in an intron, Mottagui-Tabar et al (10) have reported that individuals carrying the 11482A allele had significantly reduced PLIN protein expression in their adipocytes, linking this polymorphism to a metabolic situation similar to that reported in PLIN-null mice.

Accordingly, we found that, the PLIN 11482G>A/14995A>T polymorphisms, in high linkage disequilibrium, strongly modulate the association between diet and HOMA-IR in Asian women. Homozygotes for the variant allele have highest HOMA-IR when consuming high SFA and low CHO diets.

TABLE 1
Descriptive characteristics¹ of Singapore population by
gender and ethnic group

| | CHINESE | MALAY | INDIAN |
|---|---|---|---|
| MEN | (n = 1263) | (n = 360) | (n = 286) |
| Age (years) | 38.2 ± 12.3 | 39.6 ± 12.7 | 41.3 ± 12.1 |
| BMI (kg/m²) | 23.5 ± 3.7 | 24.7 ± 4.0 | 24.6 ± 4.0 |
| Total cholesterol (mmol/l) | 5.52 ± 1.04 | 5.88 ± 1.13 | 5.72 ± 1.17 |
| LDL-C (mmol/l) | 3.54 ± 0.95 | 3.95 ± 1.02 | 3.88 ± 1.08 |
| HDL-C (mmol/l) | 1.27 ± 0.32 | 1.15 ± 0.28 | 1.06 ± 0.29 |
| Fasting TAG (mmol//l) | 1.69 ± 1.55 | 2.00 ± 1.59 | 2.08 ± 1.78 |
| Current smoker (%) | 298 (23.4) | 162 (45.0) | 87 (30.4) |
| Alcohol user (%) | 749 (59.3) | 44 (12.2) | 149 (52.1) |
| Diabetes* (%) | | | |
| Subjects previously diagnosed | 38 (3.0) | 15 (4.2) | 24 (8.4) |
| Total (Newly diagnosed | 91 (7.2) | 39 (10.9) | 52 (18.2) |
| and known) | | | |
| PLIN polymorphisms | | | |
| 11482G > A (A allele | 0.42 | 0.43 | 0.31 |
| frequency) | | | |
| 14995A > T (T allele | 0.44 | 0.44 | 0.34 |
| frequency) | | | |
| WOMEN | (n = 1500) | (n = 386) | (n = 312) |
| Age (years) | 37.8 ± 12.2 | 38.4 ± 12.7 | 40.0 ± 12.1 |
| BMI (kg/m²) | 22.1 ± 3.6 | 26.3 ± 5.6 | 25.6 ± 5.0 |
| TC (mmol/l) | 5.33 ± 1.05 | 5.73 ± 1.17 | 5.33 ± 1.03 |
| LDL-C (mmol/l) | 3.24 ± 0.93 | 3.75 ± 1.13 | 3.53 ± 0.96 |
| HDL-C (mmol/l) | 1.56 ± 0.37 | 1.44 ± 0.33 | 1.23 ± 0.31 |
| Fasting TAG (mmol//l) | 1.16 ± 0.75 | 1.39 ± 0.88 | 1.33 ± 0.68 |
| Current smoker (%) | 45 (3.0) | 15 (3.8) | 1 (0.3) |
| Alcohol user (%) | 494 (32.9) | 12 (3.1) | 55 (17.6) |
| Diabetes* (%) | | | |
| Subjects previously diagnosed | 26 (1.7) | 22 (5.7) | 22 (7.1) |
| Total (Newly diagnosed | 99 (6.6) | 57 (14.8) | 54 (17.4) |
| and known) | | | |
| PLIN polymorphisms | | | |
| 11482G > A (A allele | 0.41 | 0.45 | 0.34 |
| frequency) | | | |
| 14995A > T (T allele | 0.44 | 0.45 | 0.38 |
| frequency) | | | |

¹Continuous variables were presented as mean ± standard deviation, while categorical variables were presented as the number of cases and percentages of prevalence.
LDL-C: low-density lipoprotein cholesterol. HDL-C: high-density lipoprotein cholesterol.
TAG: triacylglycerides.
*Prevalence of diabetes was estimated considering both the known diabetes and the newly diagnosed diabetes accorging to the biochemical analysis and the World Health Organization criteria.

TABLE 2
Adjusted insulin-resistance measures by PLIN 11482G > A genotypes, gender, and ethnics in Singapore population.

| | Men | | | | Women | | | |
|---|---|---|---|---|---|---|---|---|
| | GG | GA | AA | P¹ | GG | GA | AA | P¹ |
| CHINESE | (n = 436) | (n = 605) | (n = 222) | | (n = 526) | (n = 728) | (n = 246) | |
| Fasting glucose (mmol/L) | 5.8 (0.1) | 5.8 (0.1) | 5.7 (0.1) | 0.597 | 5.5 (0.1) | 5.5 (0.1) | 5.5 (0.1) | 0.435 |
| OGTT² (mmol/L) | 6.6 (0.1) | 6.5 (0.1) | 6.6 (0.2) | 0.924 | 6.6 (0.1) | 6.9 (0.1) | 6.7 (0.2) | 0.213 |
| Fasting insulin (mU/L) | 8.6 (0.8) | 7.5 (0.7) | 7.3 (1.1) | 0.400 | 7.1 (0.2) | 7.1 (0.1) | 6.9 (0.3) | 0.807 |
| Post-challenge insulin² (mU/L) | 58 (3) | 55 (2) | 51 (4) | 0.600 | 61 (2) | 63 (2) | 57.1 (3.3) | 0.621 |
| HOMA-IR | 1.8 (0.1) | 1.9 (0.0) | 1.8 (0.1) | 0.342 | 1.7 (0.1) | 1.7 (0.0) | 1.7 (0.1) | 0.672 |
| MALAY | (n = 116) | (n = 177) | (n = 67) | | (n = 113) | (n = 202) | (n = 71) | |
| Fasting glucose (mmol/L) | 6.2 (0.1) | 6.0 (0.1) | 6.1 (0.2) | 0.354 | 6.3 (0.2) | 6.2 (0.1) | 5.7 (0.2) | 0.171 |
| OGTT² (mmol/L) | 7.5 (0.3) | 7.1 (0.2) | 7.3 (0.4) | 0.818 | 7.6 (0.3) | 7.9 (0.2) | 7.1 (0.4) | 0.348 |
| Fasting insulin (mU/L) | 7.7 (0.5) | 8.2 (0.4) | 8.6 (0.6) | 0.563 | 10.0 (0.7) | 9.6 (0.5) | 9.2 (0.8) | 0.450 |
| Post-challenge insulin² (mU/L) | 65 (7) | 73 (5) | 64 (9) | 0.546 | 75 (6) | 77 (4) | 75 (7) | 0.777 |
| HOMA-IR | 2.2 (0.2) | 2.1 (0.1) | 2.3 (0.2) | 0.520 | 2.6 (0.2) | 2.6 (0.2) | 2.3 (0.3) | 0.429 |
| INDIAN | (n = 135) | (n = 126) | (n = 25) | | (n = 128) | (n = 154) | (n = 30) | |
| Fasting glucose (mmol/L) | 6.2 (0.2) | 6.7 (0.2) | 6.3 (0.4) | 0.107 | 6.1 (0.2) | 6.1 (0.1) | 6.1 (0.3) | 0.978 |
| OGTT² (mmol/L) | 7.2 (0.3) | 8.2 (0.3) | 6.2 (0.8) | 0.083 | 7.5 (0.3) | 7.9 (0.3) | 7.9 (0.7) | 0.564 |
| Fasting insulin (mU/L) | 10.6 (0.8) | 10.9 (0.8) | 8.5 (1.8) | 0.473 | 11.4 (0.5) | 10.7 (0.5) | 11.1 (1.0) | 0.772 |
| Post-challenge insulin² (mU/L) | 87 (8) | 91 (8) | 68 (20) | 0.470 | 92 (7) | 90 (6) | 99 (14) | 0.490 |
| HOMA-IR | 2.9 (0.3) | 3.0 (0.3) | 1.9 (0.7) | 0.282 | 2.9 (0.2) | 2.8 (0.2) | 3.1 (0.4) | 0.545 |

Values are adjusted means and (SEM).
¹Test for homogenesis across genotypes. Means were adjusted for age, BMI, smoking, alcohol, physical activity and diabetes status.
²These tests were not carried out in diabetic subjects on medication (68 men: 34 Chinese, 13 Malays and 21 Asian Indians; and 65 women: 24 Chinese, 22 Malays and 22 Asian Indians.
OGTT: oral glucose tolerance test (2h);
HOMA-IR: Homeostasis model assessment-insulin resistance.

TABLE 3
Characteristic of dietary intakes of fat, fatty acids, carbohydrate and protein by ethnics and gender
in the Singaporean population

| | Chinese | | Malay | | Indian | |
|---|---|---|---|---|---|---|
| | Men (n = 597) | Women (n = 714) | Men (n = 217) | Women (n = 239) | Men (n = 187) | Women (n = 200) |
| | Mean (SD) | Mean (SD) | Mean (SD) | Mean (SD) | Mean (SD) | Mean (SD) |
| Energy (kJ) | 9808 (3281) | 7572 (2583) | 10447 (4643) | 7902 (2598) | 10022 (3171) | 8788 (3175) |
| Total fat (g) | 70.8 (31.5) | 55.3 (25.2) | 81.4 (49.4) | 59.8 (25.7) | 74.3 (31.2) | 68.1 (33.9) |
| Total fat (% energy) | 26.6 (5.3) | 26.9 (5.4) | 28.1 (6.8) | 27.8 (5.2) | 27.5 (5.5) | 28.5 (5.8) |
| SFA (% energy) | 10.5 (2.5) | 10.1 (2.5) | 12.1 (3.6) | 11.5 (2.8) | 11.4 (3.0) | 11.7 (3.0) |
| PUFA (% energy) | 4.9 (1.7) | 5.6 (2.2) | 4.5 (1.8) | 5.2 (2.2) | 5.5 (2.2) | 6.3 (3.0) |
| MUFA (% energy) | 9.3 (2.3) | 9.3 (2.4) | 9.2 (2.7) | 9.1 (2.4) | 8.3 (2.3) | 8.2 (2.3) |
| Protein (g) | 78.2 (27.2) | 63.5 (23.2) | 75.5 (37.1) | 60.7 (20.2) | 71.7 (25.3) | 63.1 (22.7) |
| Protein (% energy) | 13.4 (1.7) | 14.0 (1.9) | 12.0 (1.7) | 13.0 (2.0) | 12.0 (1.6) | 12.1 (1.6) |
| Carbohydrate (g) | 349.3 (114.9) | 264.9 (86.0) | 366.2 (143.9) | 277.5 (88.9) | 360.6 (110.5) | 309.2 (106.7) |
| % Carbohydrate | 60.0 (6.3) | 59.1 (6.4) | 59.9 (7.5) | 59.2 (5.8) | 60.6 (6.2) | 59.4 (6.2) |

All values are means.
SD: Standard deviation
SFA: Saturated fatty acids;
PUFA: Polyunsaturated fatty acids;
MUFA: Monounsaturated fatty acids.

TABLE 4
Interactions between PLIN 11482G > A genotypes and intakes of total fat, SFA,
and CHO in determining plasma insulin-resistance related measures in Singapore
women (Chinese, Malay and Asian Indian). Adjusted means and (SEM).

| | | Tertiles | | | Trend | Interaction |
|---|---|---|---|---|---|---|
| | Genotypes¹ | 1 (n = 384) | 2 (n = 385) | 3 (n = 384) | P² | P³ |
| Total fat (tertiles of intake, %) | | 7.1 to 25.2 | 25.2 to 29.8 | 29.8 to 54.1 | | |
| Fasting insulin (mU/l) | GG + GA | 8.62 (0.31) | 8.16 (0.29) | 8.31 (0.30) | 0.824 | 0.010 |
| | AA | 7.53 (0.62) | 8.10 (0.62) | 9.31 (0.59) | 0.134 | |
| HOMA-IR | GG + GA | 2.23 (0.09) | 2.10 (0.08) | 2.19 (0.08) | 0.737 | 0.007 |
| | AA | 1.89 (0.18) | 2.10 (0.17) | 2.41 (0.17) | 0.104 | |
| SFA (tertiles of intake, %) | | 3.1 to 9.4 | 9.4 to 11.8 | 11.8 to 19.0 | | |
| Fasting glucose (mmol/l) | GG + GA | 5.62 (0.07) | 5.60 (0.07) | 5.66 (0.06) | 0.818 | 0.004 |
| | AA | 5.35 (0.12) | 5.55 (0.12) | 5.82 (0.12) | 0.014 | |
| Fasting insulin (mU/l) | GG + GA | 8.49 (0.31) | 8.37 (0.33) | 8.24 (0.28) | 0.938 | 0.004 |
| | AA | 7.50 (0.60) | 7.52 (0.61) | 9.82 (0.60) | 0.030 | |
| HOMA-IR | GG + GA | 2.19 (0.09) | 2.16 (0.10) | 2.17 (0.08) | 0.978 | 0.003 |
| | AA | 1.79 (0.17) | 1.90 (0.17) | 2.65 (0.17) | 0.006 | |
| CHO (tertiles of intake, %) | | 34.5 to 56.3 | 56.3 to 61.7 | 61.7 to 84.5 | | |
| Fasting insulin (mU/l) | GG + GA | 8.07 (0.30) | 8.43 (0.30) | 8.55 (0.30) | 0.244 | 0.007 |
| | AA | 9.10 (0.61) | 8.54 (0.57) | 7.32 (0.64) | 0.042 | |
| HOMA-IR | GG + GA | 2.12 (0.08) | 2.06 (0.09) | 2.18 (0.09) | 0.338 | 0.004 |
| | AA | 2.35 (0.17) | 2.16 (0.16) | 1.89 (0.18) | 0.046 | |

¹The distribution of PLIN 11482G > A genotypes in these 1153 women was: 424 GG, 552 GA and 177 AA
²P for lineal trend was estimated by comparing adjusted means of insulin-resistance variables across tertiles of macronutrient intake depending on the genotype group (expressed as % of energy). Means were adjusted for ethnicity, age, BMI, cigarette smoking, alcohol consumption, physical activity, diabetes status and energy intake.
³P value for the interaction term between the PLIN 11482G > A polymorphism and tertiles of the corresponding macronutrient intake in the hierarchial multivariate regression model.
SFA: saturated fat;
CHO: carbohydrate;
HOMA-IR: Homeostasis model assessment-insulin resistance.

### REFERENCES

The references cited herein and throughout the specification are herein incorporated by reference in their entirety.
1. Zimmet P, Alberti K G, Shaw J. Related Articles, Links Abstract Global and societal implications of the diabetes epidemic. Nature. 2001; 414:782-7.
2. DeFronzo R A, Ferrannini E. Insulin resistance. A multifaceted syndrome responsible for NIDDM, obesity, hypertension, dyslipidemia, and atherosclerotic cardiovascular disease. Diabetes Care 1991; 14:173-94.
3. Eckel R H, Grundy S M, Zimmet P Z. The metabolic syndrome. Lancet. 2005; 365:1415-28.
4. Stem M P. Strategies and prospects for finding insulin resistance genes. J Clin Invest 2000; 106:323-27.
5. Menzaghi C, Ercolino T, Di Paola R et al. A haplotype at the adiponectin locus is associated with obesity and other features of the insulin resistance syndrome. Diabetes 2002; 51:2306-12.
6. Londos C, Gruia-Gray J, Brasaemle D L et al. Perilipin: possible roles in structure and metabolism of intracellular neutral lipids in adipocytes and steroidogenic cells. Int J Obes Relat Metab Disord 1996; 20 Suppl 3: 97-101.
7. Martinez-Botas J, Anderson J B, Tessier D et al. Absence of perilipin results in leanness and reverses obesity in Lepr(db/db) mice. Nat Genet 2000; 26: 474-9.
8. Sztalryd C, Xu G, Dorward H et al. Perilipin A is essential for the translocation of hormone-sensitive lipase during lipolytic activation. J Cell Biol 2003; 161: 1093-103.
9. Tansey J T, Sztalryd C, Gruia-Gray J et al. Perilipin ablation results in a lean mouse with aberrant adipocyte lipolysis, enhanced leptin production, and resistance to diet-induced obesity. Proc Natl Acad Sci USA 2001; 98: 6494-9.
10. Mottagui-Tabar S. Ryden M, Lofgren P et al. Evidence for an important role of perilipin in the regulation of human adipocyte lipolysis. Diabetologia 2003; 46: 789-97.
11. Qi L, Corella D, Sorli J V, et al. Genetic variation at the perilipin (PLIN) locus is associated with obesity-related phenotypes in White women. Clin Genet 2004; 66:299-310.
12. Qi L, Shen H, Larson I, et al. Gender-specific association of a perilipin gene haplotype with obesity risk in a white population. Obes Res 2004; 12:1758-65.
13. Qi L, Shen H, Tai E S, et al. Intragenic linkage disequilibrium structure of the human perilipin gene (PLIN) and haplotype association with increased obesity risk in a multi-ethnic Asian population. J Mol Med 2005; 83: 448-56.
14. Corella D, Qi L, Sorli J V, et al. Obese subjects carrying the 11482G>A polymorphism at the perilipin (PLIN) locus are resistant to weight loss following dietary energy restriction. J Clin Endocrinol Metab 2005 (in press).
15. Cutter J, Tan B Y, Chew S K. Levels of cardiovascular disease risk factors in Singapore following a national intervention programme. Bull World Health Organ 2001; 79: 908-15.
16. Tai E S, Corella D, Deurenberg-Yap M et al. Dietary fat interacts with the −514C>T polymorphism in the hepatic lipase gene promoter on plasma lipid profiles in a multiethnic Asian population: the 1998 Singapore National Health Survey. J Nutr 2003; 133: 3399-408.
17. Deurenberg-Yap M, Li T, Tan W L et al. Can dietary factors explain differences in serum cholesterol profiles among different ethnic groups (Chinese, Malays and Indians) in Singapore? Asia Pac J Clin-Nutr 2001; 10: 39-45.
18. Tai E S, Ordovas J M, Corella D et al. The TaqIB and −629C>A polymorphisms at the cholesteryl ester transfer protein locus: associations with lipid levels in a multiethnic population. The 1998 Singapore National Health Survey. Clin Genet 2003; 63: 19-30.
19. Matthews D R, Hosker J P, Rudenski A S et al. Homeostasis model assessment: insulin resistance and beta-cell function from fasting plasma glucose and insulin concentrations in man. Diabetologia 1985; 28: 412-9.
20. Alberti K G, Zimmet P Z. Definition, diagnosis and classification of diabetes mellitus and its complications. Part 1: diagnosis and classification of diabetes mellitus provisional report of a WHO consultation. Diabet Med 1998; 15: 539-53.
21. Astrup A, Meinert Larsen T, Harper A. Atkins and other low-carbohydrate diets: hoax or an effective tool for weight loss? Lancet 2004; 364:897-9.
22. Golay A, Eigenheer C, Morel Y, Ku jawski P, Lelunann T, de Tonnac N. Weight-loss with low or high carbohydrate diet? Int J Obes Relat Metab Disord 1996; 20: 1067-72.
23. Bravata D M, Sanders L, Huang J, et al. Efficacy and safety of low-carbohydrate diets: a systematic review. JAMA. 2003; 289:1837-50.
24. Moore H, Summerbell C, Hooper L, et al. Dietary advice for treatment of type 2 diabetes mellitus in adults. Cochrane Database Syst Rev. 2004; (3):CD004097.
25. Schwenke D C. Insulin resistance, low-fat diets, and low-carbohydrate diets: time to test new menus. Curr Opin Lipidol. 2005; 16:55-60.
26. Schoeller D A, Buchholz A C. Energetics of obesity and weight control: does diet composition matter? J Am Diet Assoc. 2005; 105:S24-8.
27. Crowe T C. Sfatey of low-carbohydrate diets. Obesity Rev 2005; 6: 235-45.
28. Boden G, Sargrad K, Homko C, Mozzoli M, Stein T P. Effect of a low-carbohydrate diet on appetite, blood glucose levels, and insulin resistance in obese patients with type 2 diabetes. Ann Intern Med. 2005; 142:403-11.
29. Stem L, Iqbal N, Seshadri P, et al. The effects of low-carbohydrate versus conventional weight loss diets in severely obese adults: one-year follow-up of a randomized trial. Ann Intern Med 2004; 140:778-85.
30. Ordovas J M, Corella D. Nutritional genomics. Annu Rev Genomics Hum Genet. 2004; 5:71-118.
31. Luan J, Browne P O, Harding A H, et al. Evidence for gene-nutrient interaction at the PPARgamma locus. Diabetes 2001; 50:686-9.
32. Robitaille J, Despres J P, Perusse L, Vohl M C. The PPAR-gamma P12A polymorphism modulates the relationship between dietary fat intake and components of the metabolic syndrome: results from the Quebec Family Study. Clin Genet. 2003; 63:109-16.
33. Tai E S, Corella D, Deurenberg-Yap M, et al. Differential effects of the C1431T and Pro12Ala PPARgamma gene variants on plasma lipids and diabetes risk in an Asian population. J Lipid Res. 2004; 45:674-85.
34. Ravussin E, Smith S R. Increased fat intake, impaired fat oxidation, and failure of fat cell proliferation result in ectopic fat storage, insulin resistance, and type 2 diabetes mellitus. Ann N Y Acad Sci 2002; 967: 363-78.
35. Wang H, Chu W S, Hemphill C, Elbein S C. Human resistin gene: molecular scanning and evaluation of association with insulin sensitivity and type 2 diabetes in Caucasians. J Clin Endocrinol Metab 2002; 87: 2520-4.
36. Riccardi G, Giacco R, Rivellese AA. Dietary fat, insulin sensitivity and the metabolic syndrome. Clin Nutr. 2004; 23:447-56.
37. Vessy B, Uusitupa M, Hennansen K, et al. Substituting dietary saturated for monounsaturated fat impairs insulin sensitivity in healthy men and women: the KANWU study. Diabetologia 2001; 44:312-9.
38. DeFronzo R, Tobin J, Andres R. Glucose clamp technique: a method for quantifying insulin secretion and resistance. Am J Physiol 1979: 237: E214-23.
39. Bonora E, Targher G, Alberiche M et al. Homeostasis model assessment closely mirrors the glucose clamp technique in the assessment of insulin sensitivity: studies in subjects with various degrees of glucose tolerance and insulin sensitivity. Diabetes Care. 2000; 23:57-63.
40. McAuley K A, Williams S M, Mann J I, et al. Diagnosing insulin resistance in the general population. Diabetes Care 2001; 24:460-4.
41. McKeown N M, Meigs J B, Liu S, Saltzman E, Wilson P W, Jacques P F. Carbohydrate nutrition, insulin resistance, and the prevalence of the metabolic syndrome in the Framingham Offspring Cohort. Diabetes Care. 2004; 27:538-46.
42. Lau C, Faerch K, Glumer C, et al. Dietary glycemic index, glycemic load, fiber, simple sugars, and insulin resistance: the Inter99 study. Diabetes Care. 2005; 28:1397-403.
43. Ludwig D S, Pereira M A, Kroenke C H, et al. Dietary fiber, weight gain, and cardiovascular disease risk factors in young adults. JAMA 1999; 282:1539-46.
44. Marshall J A, Bessesen D H, Hamman R F. High saturated fat and low starch and fibre are associated with hyperinsulinaemia in a non-diabetic population: the San Luis Valley Diabetes Study. Diabetologia 1997; 40:430-8.
45. Salmeron J, Manson J E, Stampfer M J, Colditz G A, Wing A L, Willett W C. Dietary fiber, glycemic load, and risk of non-insulin-dependent diabetes mellitus in women. JAMA 1997; 277:472-7.
46. Salmeron J, Ascherio A, Rimm E B, et al. Dietary fiber, glycemic load, and risk of NIDDM in men. Diabetes Care 1997; 20:545-50.
47. Meyer K A, Kushi L H, Jacobs D R Jr, Slavin J, Sellers T A, Folsom A R. Carbohydrates, dietary fiber, and incident type 2 diabetes in older women. Am J Clin Nutr 2000; 71:921-30.
48. Jenkins D J, Kendall C W, Augustin L S, et al. Glycemic index: overview of implications in health and disease. Am J Clin Nutr 2002; 76:266 S-73S.
49. Pi-Sunyer FX. Glycemic index and disease. Am J Clin Nutr 2002; 76:290 S-8S.
50. German J B, Dillard C J. Saturated fats: what dietary intake? Am J Clin Nutr. 2004; 80:550-9.
51. McAuley K A, et al. Comparison of high-fat and high-protein diets with a high-carbohydrate diet in insulin-resistant obese women. Diabetologia 2005; 48:8-16.
52. Mittendorfer B. Insulin resistance: sex matters. Curr Opin Clin Nutr Metab Care 2005; 8:367-72.
53. Saha P K, Kojima H, et al. Metabolic adaptations in the absence of perilipin: increased beta-oxidation and decreased hepatic glucose production associated with peripheral insulin resistance but normal glucose tolerance in perilipin-null mice. J Biol Chem 2004; 279:35150-8.

I claim:

1. A method for screening for females of Asian origin who are at risk of developing metabolic syndrome and diseases associated with metabolic syndrome, when consuming high-saturated fatty acid/low-carbohydrate containing diet, the method comprising determining PLIN 11482 locus genotype in a biological sample taken from a female of Asian origin, wherein presence of two PLIN 11482A alleles or alleles that are found to be in tight linkage disequilibrium with PLIN 11482A allele, is indicative of the female who is at risk of developing metabolic syndrome and diseases associated with metabolic syndrome, when consuming high-saturated fatty acid/low-carbohydrate diet.

2. A kit providing dietary advice to an individual comprising:
a) a system for genotyping two PLIN 11482 locus for A and/or G allele from a biological sample; and
b) instructions that the kit be used in connection with screening for females of Asian origins and that if two PLIN 11482A alleles are detected in the biological sample from a female of Asian origin, the individual is advised to limit consumption of saturated fatty acids and increase consumption of carbohydrates, to avoid or manage metabolic syndrome or conditions associated with metabolic syndrome.

3. A kit providing dietary advice to an individual comprising:
a) a system for genotyping PLIN 11482 locus from a biological sample taken from a female of Asian origin; and
b) instructions that if two PLIN 11482A alleles are detected, the individual is advised to limit or avoid consumption of foods containing saturated fatty acids and increase consumption of foods rich in carbohydrates.

4. The kit of claim 3, wherein the system for genotyping PLIN 11482 locus comprises attached nucleic acid probes on a solid surface.

5. The kit of claim 4, wherein the solid surface is selected from chips and beads.

6. A kit for screening for females of Asian origin at risk of metabolic syndrome and health conditions associated with metabolic syndrome, when exposed to a diet high in saturated fatty acids and low in carbohydrates, wherein the kit comprises a plurality of isolated oligonucleotides, the oligonucleotides corresponding to no more than about 100 polymorphisms, wherein at least one of the polymorphisms is PLIN 11482, and a guideline that indicates that if two PLIN 11482A alleles are detected in a biological sample from an individual, the individual should be advised to avoid a diet high in saturated fatty acids and low in carbohydrates.

7. The kit of claim 6, wherein the plurality of isolated oligonucleotides are attached on a solid surface.

8. The kit of claim 6, wherein the sold surface is selected from nucleic acid chips and beads.

9. A method for creating diet advice comprising:
a) providing a service to screen PLIN 11482A/G alleles in a biological sample from a female of Asian origin;
b) providing a service to create diet advice with decreased saturated fatty acids and increased carbohydrates for the female whose sample contains two PLIN 11482A; and
c) delivering the diet advice to the individual.

10. A method for creating diet advice comprising:
a) providing a service to create diet advice with decreased saturated fatty acids and increased carbohydrates for females of Asian origin whose biological sample contains two PLIN 11482A alleles; and
b) delivering the diet advice to the individual.

11. A method to direct dietary advice to a female of Asian origin, wherein PLIN11482 genotype is utilized for the direction of dietary advice, and wherein homozygosity for PLIN11482A allele is used to indicate that the individual being homozygous for the PLIN11482A allele is to avoid high fat/low carbohydrate diet, and/or is to prefer eating low-fat/high carbohydrate diet.

12. The method of claims 1, 9, 10 or 11, wherein the female of Asian origin is selected from women of Chinese, Malay or Indian origin.
